(19) United States
(12) Patent Application Publication
(10) Pub. No.: US 2023/0149352 A1
(43) Pub. Date: May 18, 2023

(54) USES OF APREMILAST

(71) Applicant: APRAMITHA INNOVATIONS PRIVATE LIMITED, Hyderabad, Telangana (IN)

(72) Inventor: Venkata Nookaraju SREEDHARALA, Hyderabad (IN)

(73) Assignee: APRAMITHA INNOVATIONS PRIVATE LIMITED, Hyderabad, Telangana (IN)

(21) Appl. No.: 17/920,602

(22) PCT Filed: Apr. 21, 2021
(86) PCT No.: PCT/IN2021/050392

§ 371 Date: Oct. 21, 2022

(30) Foreign Application Priority Data
Apr. 22, 2020 (IN) 202041017330

(51) Int. Cl. A61K 31/4035; A61K 9/00; A61P 17/00

(52) U.S. Cl. CPC A61K 31/4035; A61K 9/0014; A61P 17/00

(57) ABSTRACT

The present invention is related to methods of treating, preventing, correcting and / or managing non-psoriatic skin diseases or disorders, characterized by dry, hardened, scaly or flaky skin and fissures. The present invention also provides various topical administration of therapeutically or prophylactically effective amount of an Apremilast for treating, preventing, correcting and/ or managing non-psoriatic skin diseases or disorders, characterized by dry, hardened, scaly or flaky skin and fissures. The present invention further relates to a method of treating, preventing, and managing the conditions that cause cytokine storm, by administration of Apremilast.

### CROSS REFERENCE

This application claims priority to an Indian provisional patent application no. 202041017330, filed Apr. 22, 2020, the contents of which are incorporated herein by reference in their entireties.

### FIELD OF THE INVENTION

The present invention relates to methods of treating, preventing, correcting and / or managing non-psoriatic skin diseases or disorders, characterized by dry, hardened, scaly or flaky skin and fissures. Specifically, the present invention relates to stable topical compositions comprising Apremilast, for healing non-psoriatic skin diseases or disorders, particularly epidermal cracks or fissures on the soles of feet, primarily on heels. Also disclosed are methods of preparation and use of stable and effective topical pharmaceutical compositions of Apremilast for use in the present invention. The present invention also provides method of treating, preventing, and managing the conditions that cause cytokine storm such as influenza, respiratory tract infections and pneumonia caused by viruses, specifically, those belonging to the Paramyxoviridae, Orthomyxoviridae, Flaviviridae, Picomaviridae, and Coronaviridae family, by administration of Apremilast.

### BACKGROUND OF THE INVENTION

Epidermal cracks or fissures, particularly on the heel or toes are common foot problem. In most cases the problem is merely a nuisance and unattractive to look at, however, when the cracks or fissures become deep, standing and walking or any pressure placed on the heel can be painful. Dry thickened skin (corn and callus) around the rim of the heel or toe is the very first step towards cracking. Increased pressure on the fat pad under the heel causes it to expand sideways, leading to splitting or cracking of the callus. Initially, small cracks over the callus are visible, if left untreated and as more pressure is placed on the heel, these cracks become deeper and eventually walking and standing will be painful. The cracks may be so deep that they begin to bleed and may lead to unwanted infections that further complicate condition.

In severe cases, cracked heels can become infected, and lead to cellulitis. This must be treated with the elevation of the area, debridement of dead tissue, and antibiotics. Cracked heels are of particular concern for diabetic patients, who may suffer neuropathic damage (loss of feeling, particularly of the feet), as the fissures may lead to diabetic foot ulcers.

The best form of treatment for cracked heel is to prevent cracks from occurring in the first place. This can be achieved by a combination of exfoliation, moisturizing, and avoidance of the environmental conditions that seem to exacerbate the condition.

Special heel balms and moisturizers are available that contain descaling (keratolytic) or water-retaining (humectant) agents, such as urea, salicylic acid, alpha-hydroxy acids and saccharide isomerate.

Prior art formulations, developed to assist in the various skin conditions including cracked heels, can be found in U.S. Pat. No. 10,143,716, and U.S. Pat. No. 9,433,572, wherein topical compositions comprising fruit extracts are disclosed. Though these formulations may be adequate for their respective intended purposes, neither teaches a composition that provides healing and relief from more severe heel cracks and fissures, in particular.

U.S. Pat. No. 7,119,117 discloses Tocopherol enriched compositions for reducing non-psoriatic skin inflammations and disorders. U.S. Pat. No. 5,691,327 discloses formulations which contain, among other possible ingredients, salicylic acid, hydrocortisone, zinc oxide and boric acid in concentration ranges from 0.1% to 20%. Further, US20010005721A also discloses heel cream compositions consisting of a combination of a safe and effective amount of salicylic acid and hydrocortisone.

However, these disclosures do not teach nor enable a composition for healing and preventing cracked heels in particular, nor do they teach specific ranges of respective ingredients that have testably been most therapeutic. Further, the best known treatment options only target the symptoms of the disease or disorder and simply provide short term improvement. There are also undesirable side effects. Thus, there is still a need for safe and effective methods for the treatment and management of cracked heels.

Similarly, for pathological conditions that cause cytokine storm such as acute viral infections causing influenza, respiratory tract infections and pneumonia, current antiviral treatments work with varying degrees of success when administered shortly after symptom onset. While the infectious agent plays a role in disease and pathogenesis, the overzealous immune response to the infection also significantly contributes to the etiology of severe respiratory illnesses. Additional medical therapies are needed which could be beneficial that target multiple aspects of a respiratory infection, including, for example, mucus overproduction, airway hyper-responsiveness, and that could also inhibit replication of the underlying infectious agent.

### SUMMARY OF THE INVENTION

An object of the present invention is to provide methods of treating, preventing, correcting and / or managing non-psoriatic skin diseases or disorders, characterized by dry, hardened, scaly or flaky skin and fissures.

In one embodiment the present invention comprises topical administration of therapeutically or prophylactically effective amount of an Apremilast for treating, preventing, correcting and / or managing non-psoriatic skin diseases or disorders, characterized by dry, hardened, scaly or flaky skin and fissures.

Another embodiment of the present invention provides stable topical compositions comprising therapeutically effective amount of Apremilast and pharmaceutically acceptable carriers, for healing non-psoriatic skin diseases or disorders, particularly epidermal cracks or fissures on the soles of feet, primarily on heels.

In yet another embodiment, the present invention is directed to methods of treating non-psoriatic skin diseases or disorders, characterized by dry, hardened, scaly or flaky skin and fissures, by topical application to an affected epidermal area of a subject a topical dosage form comprising Apremilast; and continuing the administration until symptoms of the said disorder are abated.

Another object of the present invention is to provide a simple, safe and commercially viable process for preparation of a topical composition of Apremilast that is sufficiently stable to provide an acceptable shelf life.

Yet another objective of the present invention is to provide methods of treating, preventing, and managing pathological conditions that cause cytokine storm such as influenza, respiratory tract infections and pneumonia caused by viruses, specifically, those belonging to the Paramyxoviridae, Orthomyxoviridae, Flaviviridae, Picomaviridae, and Coronaviridae family.

In one embodiment the present invention comprises administration of therapeutically or prophylactically effective amount of an Apremilast for treating, preventing, and managing pathological conditions that cause cytokine storm such as influenza, respiratory tract infections and pneumonia caused by viruses, specifically, those belonging to the Paramyxoviridae, Orthomyxoviridae, Flaviviridae, Picornaviridae, and Coronaviridae family.

### FIGURES OF THE INVENTION

FIG. 1 shows the effect of the topical composition on affected toe and its stages during treatment as:

FIG. 1(A): Showing complete feet picture before initiating the treatment showing cracks around the rim of the heels (101) and hardened skin on the toe (102).

FIG. 1(B): Showing complete feet picture after 1^{st} day treatment with the topical gel of the current invention.

FIG. 1(C): Showing complete feet picture after 2ⁿ^{d} day treatment with the topical gel of the current invention clearly demonstrating the reduction or disappearance of the cracks around the rim of the heels (202) and softened toe skin post treatment (201).

FIG. 2: Showing comparison of fissure foot before and after 25 days of treatment with Topical Apremilast gel in 36 year male patient.

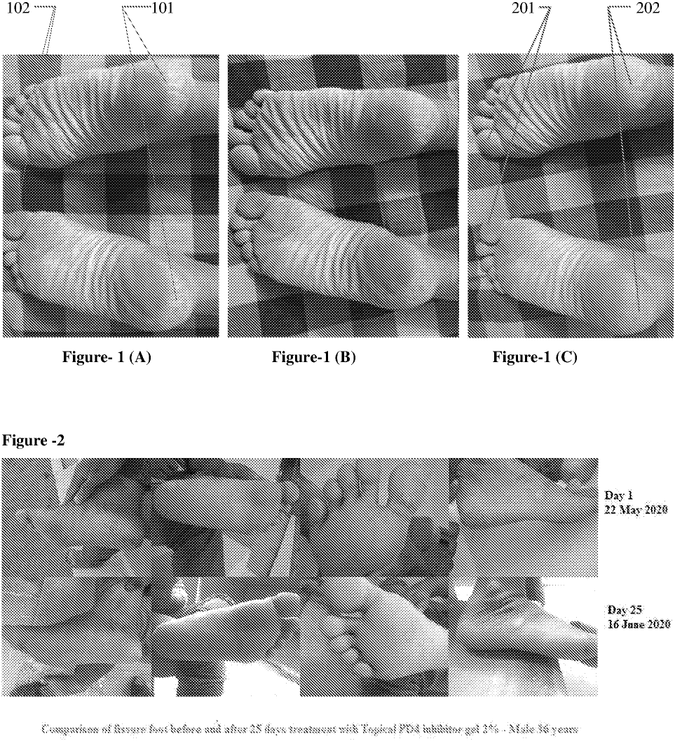

### DETAILED DESCRIPTION OF THE INVENTION

The present invention relates to methods of treating, preventing, correcting and / or managing non-psoriatic skin diseases or disorders, characterized by dry, hardened, scaly or flaky skin and fissures, by topical administration of therapeutically or prophylactically effective amount of Apremilast.

Apremilast, an orally administered small molecule inhibitor of phosphodiesterase 4 (PDE4), is chemically N- [2-[(1S) -1- (3-ethoxy-4-methoxyphenyl) -2- (methylsulfonyl) ethyl] -2,3-dihydro-1,3-dioxo.-1H-isoin-dol-4-yl] acetamide.

Apremilast has been licensed by the US Food and Drug Administration for the management of active psoriatic arthritis & moderate to severe plaque psoriasis, & is currently, available from Celgene Corp. only as an oral formulation sold under the trademark OTEZLA. Otezla® tablets are supplied as oral doses of 10 mg, 20 mg & 30 mg.

The inventors of the present invention have surprisingly found that Apremilast could be used to effectually treat non-psoriatic skin conditions or disorders, as well. It was found that Apremilast not only has potent effectiveness in the treatment of non-psoriatic skin infirmities, particularly epidermal cracks or fissures on the soles of feet, primarily on heels, but also provided long term softening effect on the skin whereby recurrence of the said skin infirmities could be avoided or ameliorated. Thus, the presently disclosed method provides a means of treating non-psoriatic skin diseases or disorders, characterized by dry, hardened, scaly or flaky skin and fissures; by administering Apremilast in suitable topical dosage forms.

The present invention provides stable topical compositions comprising therapeutically effective amount of Apremilast and pharmaceutically acceptable carriers, for healing non-psoriatic skin diseases or disorders, particularly epidermal cracks or fissures on the soles of feet, primarily on heels and toes.

Topical formulations of the present invention include, but are not limited to, sprays, aerosols, solutions, emulsions, suspensions, lotions, creams, gels, ointments or other forms known to those skilled in the art. Specifically, the disclosed topical composition may be a cream, lotion, spray, oil, ointment, paste, dressing, solution, gel or other types of compositions that lends itself to topical application.

As used herein, “stable” refers to the physical and / or chemical stability of the active agent in a topical composition, 60% relative humidity (RH) at 25° C., 65% RH at 30° C. or 40° C. At RH 75%, the composition has less than about 10% variation in drug assay values and / or impurity content when stored for stability studies over a period of 3, 6, 12, 18, or 24 months, etc.

As used herein, “topical” means a composition intended for application to skin, nail or mucosal tissue.

As used herein, “Apremilast” includes Apremilast and its salts, polymorphs, hydrates, solvates, prodrugs, chelates and complexes.

A “therapeutically effective amount” refers to an amount needed to temporarily alleviate at least one symptom. For example, a therapeutically effective amount is an amount sufficient to treat (i.e., alleviate or reduce) at least one of dryness, hardness, flakiness of skin, cracks or fissures, and the like. The topical composition of the present invention comprises from about 0.01% w/w to about 30% w/w of Apremilast based on the total amount of the composition.

The term “pharmaceutically acceptable” means approved by a regulatory agency of the Federal or state government or listed in the U.S. Pharmacopeia or other generally recognized pharmacopeia for use in animals, and more particularly in humans.

The term “carrier” is a natural or synthetic organic or inorganic ingredient, which means an ingredient that, in combination with the active ingredient, facilitates application of the composition. Suitable carrier materials include any of the carriers or excipients commonly used as a base for solutions, dispersions, emulsions, gels, creams, ointments, lotions, pastes, sprays or foams for topical administration. Examples include emulsifiers and inert carriers such as hydrocarbon bases, emulsifier bases, non-toxic solvents, water-soluble bases and the like. Any suitable liquid or gel carrier is well known in the art. The carrier must be capable of dissolving or dispersing an effective concentration of the active, optionally in the presence of a non-toxic surfactant. Examples include water, physiological saline, alcohols (eg, methanol, ethanol, propanol or butanol), glycerol, glycols (ethylene glycol, propylene glycol, ethoxydiglycol, etc.), polyethylene glycol (MW: 400 to 20,000, etc.), water-alcohol / glycol mixtures and the like. Suitable carriers and diluents for certain embodiments include, for example, water, saline, isotonic saline such as phosphate buffered saline, dextrose, glycerol, ethoxydiglycol, dimethyl sulfoxide (DMSO), and the like, and combinations thereof.

Suitable carriers are aqueous or oily carriers, such as white petrolatum, isopropyl myristate, lanolin or lanolin alcohol, mineral oil, fragrance or essential oil, sorbitan monooleate, cetostearyl alcohol (together or in various combinations) and detergents (polysorbate (Tweens) such as polysorbate 20, 40, 60 or 80, polyoxyl stearate, sodium lauryl sulfate, etc.) are further included. One or more carrier materials can be mixed with water to form lotions, gels, creams, semi-solid compositions, and the like. Other suitable carriers include W/O or O/W emulsions, emulsifiers and emollients and solvents (sucrose stearate, sucrose cocoate, sucrose distearate, mineral oil, propylene glycol, 2-ethyl-1,3-hexanediol, Polyoxypropylene-15-stearyl ether, water or a combination thereof). For example, a water-containing emulsion, glycerol stearate, glycerin, mineral oil, synthetic spermaceti, cetyl alcohol, or a combination thereof can be used.

The amount of carrier can be from about 5% to about 99% by total weight of the composition.

Pharmaceutically acceptable carriers includes water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassate. Includes but is not limited to tri-ethanolamine, disodium EDTA, phenoxyethanol, methylparaben, propylparaben, ethanol, titanium dioxide, biopolymers (eg, sodium hyaluronate), liposomes, nanoparticles and microparticle carriers.

In some embodiments, the composition comprises an excipient. Examples of excipients include emulsifiers, co-emulsifiers, permeation enhancers / transdermal absorption enhancers, solvents, co-solvents, emollients, propellants, antioxidants, preservatives, buffers, gelling agents or thickeners. Examples include, but are not limited to, tackifiers, polymers, plasticizers, film formers, surfactants, soothing agents, pH adjusters, solubilizers, stabilizers, water retention agents, humectants, oily bases, and the like.

As used herein, the term “emollient” refers to a substance that assists in maintaining the water content of the skin and adjusting the evaporation rate and adhesiveness of the composition. In addition, emollients provide a softening or soothing effect on the skin surface. Suitable examples of emollients include mixtures of triglyceride caprylate and triglyceride caprate (eg, Crodamol ™), triglyceride palmitate, triglyceride oleate, triglyceride caprylate, triglyceride caprate, and linoleate. Fatty acid triglyceride such as triglyceride; fatty acid ester such as isopropyl myristate, isopropyl palmitate, dibutyl adipate and dibutyl phthalate; polyhydric alcohol such as propylene glycol, butylene glycol, polyethylene glycol, glycerol and sorbitol; fatty acid such as oleic acid and stearic acid. Oils such as mineral oil, lanolin oil, coconut oil, cocoa butter, olive oil, jojoba oil and castor oil; cyclomethicone; Emissions; waxes; lecithin and mixtures thereof. The emollient is preferably selected from the group consisting of fatty acid triglycerides, fatty acid esters and polyhydric alcohols.

As used herein, “propellant” refers to a substance that promotes the discharge of the composition from a container. Suitable examples of propellants are selected from the group consisting of common non-ozone depleting hydrocarbon propellants, including propane, butane, isobutane, cyclopropane, 1,1,1,2-tetrafluoroethane, 1,1,1,2,3,3,3-heptafluoropropane, 1,1 1,1-difluoroethane, 1,1,1,3,3,3-hexafluoropropane and mixtures thereof, fluorocarbon gases, and liquefied petroleum gases.

As used herein, “emulsifier” means any of a wide variety of cationic, anionic, zwitter-ionic, and amphoteric surfactants known in the art. Examples of anionic emulsifiers include sodium lauryl sulfate, alkyl isoethionates, alkyl sulfates and alkyl ether sulfates and their salts, alkyl phosphates and alkyl ether phosphates and their salts, alkyl methyl taurates and fatty acid soaps (Alkali metal salts and sodium or potassium salts).

Examples of amphoteric and zwitter-ionic emulsifiers include those widely described as derivatives of aliphatic secondary and tertiary amines. In these derivatives, the aliphatic groups may be straight or branched, one of the aliphatic substituents containing about 8 to about 22 carbon atoms, or one of the aliphatic substituents is an anionic water soluble group (e.g., carboxy, sulfonate, Sulfate, phosphate, phosphonate). Specific examples include alkyl imino acetates, iminodialkanoates and amino alkanoates, and imidazolinium and ammonium derivatives. Other suitable amphoteric and zwitter-ionic emulsifiers include betaines, sultaines, hydroxysultaines, alkyl sarcosinates and alkanoyl sarcosinates.

Non-ionic surfactants include those broadly defined as condensates (i.e., glycosides) of long-chain alcohols (e.g., C₈-₃₀ alcohols) with sugar or starch polymers. Various sugars include, but are not limited to, glucose, fructose, mannose and galactose. Various long-chain alcohols include, but are not limited to, decyl alcohol, cetyl alcohol, stearyl alcohol, lauryl alcohol, myristyl alcohol, oleyl alcohol, and the like.

Other useful non-ionic emulsifiers include condensates of alkylene oxides and fatty acids, such as fatty acid esters of alkylene oxides. Other non-ionic surfactants are condensates of alkylene oxides with 2 moles of fatty acids (such as fatty acid di-esters of alkylene oxides).

Silicone emulsifiers are typically organically modified organopolysiloxanes, sometimes referred to as silicone surfactants. Useful silicone emulsifiers include dimethicone copolyols. These materials are polydimethylsiloxanes, but may contain polyether side chains such as polyethylene oxide chains, polypropylene oxide chains, polyether chains, mixtures of these chains, and polyether chains containing moieties derived from both ethylene oxide and propylene oxide, etc.

The amount of emulsifier can be from about 0.25% to about 45% based on the total weight of the composition.

The term “co-emulsifier” or secondary emulsifier includes polyoxyl glycerides, such as oleoyl macrogol glyceride (Labrafil® M 1944CS), linoleoyl macrogol glyceride (labrafil ( Labrafil® M 2125CS)), caprylocaproyl macrogol glyceride (Labrasol®), cetyl alcohol + ceteth-20 + steareth-20 (Emulcire® 61 WL2 659), Glyceryl stearate + PEG-75 stearate (Gelot@ 64) and mixtures thereof.

The term “Permeation enhancer” or “percutaneous absorption enhancer” is a component used to improve the penetration rate of a drug through the skin or mucous membrane, such as by temporarily reducing the impermeability of the skin or membrane. Permeation enhancers are also called “enhancers” and “absorption enhancers”. Many transdermal absorption enhancers that can be used in the present invention are known. Various useful permeation enhancers include, for example, polyols and esters, including polyethylene glycol, polyethylene glycol monolaurate, butanediol; sulfoxides, including dimethyl sulfoxide and decylmethyl sulfoxide; diethylene glycol monoethyl ether (eg, trans Ethers, including Kutol (Transcutol® P) and diethylene glycol monomethyl ether; fatty acids, including lauric acid, oleic acid, valeric acid; fatty acids, including isopropyl myristate, isopropyl palmitate, methyl propionate, and ethyl oleate Esters: urea, dimethylacetamide, dimethylformamide, 2-pyrrolidone, ethanolamine, methyl-2-pyrrolidone, diethanolamine and Terpenes, alkanones, organic acids, including salicylic acid, citric acid, and succinic acid, and mixtures thereof, and one or more surfactants may be used as permeation enhancers or agents. It can also be used as a skin absorption enhancer.

Permeation enhancers can be used in a concentration range of about 0.001 to 15%, preferably about 0.05 to 12%, more preferably about 3 to 10%, based on the total weight of the composition.

The term “preservative” means a natural or synthetic chemical substance that is added to a product to prevent the growth of microorganisms and the degradation of the product due to undesired chemical changes. Desirably, preservatives can be added to the composition to protect against the growth of potentially harmful microorganisms. Microorganisms tend to grow in the aqueous phase, but can also inhabit the hydrophobic and oily phases. Suitable preservatives for the composition of the present invention include any of methyl paraben, propyl paraben, benzyl alcohol, chlorocresol, benzalkonium chloride, cetrimonium chloride, sodium edetate, boric acid and mixtures thereof. However, the present invention is not limited to these.

The amount of preservative can be from about 0.25% to about 25% based on the total weight of the composition.

“Antioxidant” means a substance that prevents oxidation or suppresses reactions promoted by oxygen or peroxide. Antioxidants, especially lipid-soluble antioxidants, can neutralize oxygen radicals and protect the membrane by being absorbed by cell membranes. Antioxidants suitable for the compositions of the present invention include ascorbic acid (vitamin C), glutathione, lipoic acid, uric acid, carotenes, α-tocopherol (vitamin E), ubiquinol, butylated hydroxyanisole, butylated hydroxytoluene, Sodium benzoate, sodium thiosulfate, propyl gallate (PG, E310) and tert-butyl hydroquinone.

The amount of antioxidant can be from about 0.01% to about 20% based on the total weight of the composition.

“Solvent” means a component that facilitates the dissolution of a drug in the present formulation. The solvent serves to maintain a solution of the drug in the composition. Some solvents further enhance transdermal absorption of the drug and / or act as humectants. Examples of the solvent for the steroid agent include fatty acid esters of natural fatty acids, animal or plant triglycerides, medium-chain triglycerides, mono-, di- and / or tri-mixed glycerides, waxes, hydrogenated vegetable oils, and mixtures of these with water. Substances may be included. Some specific examples include castor oil, lanolin oil, C10-C18 triisocetyl citrate triglyceride, caprylic / capric triglyceride, coconut oil, corn oil, cottonseed oil, linseed oil, mink oil, olive oil, coconut oil, Examples include sunflower oil, nut oil, diethylene glycol monoethyl ether, diethylene glycol monomethyl ether, saturated paraffin oil, light or heavy mineral oil, vegetable oil or glyceride.

As used herein, a “plasticizer” is a substance that helps the present composition to form a flexible adhesive film on the skin. Suitable plasticizers include citrate esters, dimethyl isosorbide, castor oil, propylene glycol, polyethylene glycol, glycerol, oleic acid, citric acid, adipic acid, phosphate esters, fatty acid esters, glycol derivatives, hydrocarbons and derivatives thereof, Butanediol polyester, diethyl phthalate, dibutyl phthalate, chlorinated paraffins and mixtures thereof.

As used herein, “film-forming agent” is a substance that forms a stable film on a local surface when applied. Suitable film formers are acrylic acid polymers or copolymers such as methacrylic acid copolymers; cellulose derivatives such as cellulose acetate, hydroxypropylmethylcellulose, hydroxyethylcellulose, methylcellulose and ethylcellulose; polyvinyl acetate; polyvinyl alcohol; povidone; povidone vinyl acetate and mixture thereof. These film-forming agents can be partially dissolved when exposed to moisture in the skin or air, resulting in a porous film. The porosity can be improved by further adding a water-soluble additive. The water-soluble additive is preferably propylene glycol, sodium lauryl sulfate, poloxamer, polyoxyl 35 castor oil, polyoxyl 40 hydrogenated castor oil, cetomacrogol, polyethylene glycol, transcutol, or a mixture thereof.

Suitable pH adjusters are selected from the group consisting of pharmaceutically acceptable organic or inorganic acids or bases such as sodium hydroxide, tromethamine, hydrochloric acid, inorganic oxides, inorganic salts of weak acids, and mixtures thereof.

The compositions of the present invention may optionally further comprise one or more additional active ingredients such as antibacterial agents, bactericides, antifungal agents, analgesics, anti-inflammatory agents, emollients, local anesthetics, and the like.

The composition may comprise additional active agents that includes, but are not limited to: methotrexate, 6-MP, azathioprine sulfasalazine, mesalazine, olsalazine chloroquinine / hydroxychloroquine, penicylamine, aurothiomalate (intramuscular and oral), azathioprine, coxin, corticosteroids (oral, inhalation and topical injection), B-2 adrenoceptor agonists (salbutamol, terbutaline, salmeterel), xanthine (theophylline and aminophylline), cromoglycate, nedocromil, ketotifen, ipratropium and oxitropium, cyclosporine, FK506, rapamycin, mycophenolate mofetil, NSAIDs such as leflunomide, teriflunomide and ibuprofen; corticosteroids such as prednisolone; phosphodiesterase inhibitors; Nosin agonist, antithrombotic agent, complement inhibitor, adrenergic agonist, proinflammatory cytokine such as TNFa or IL-1 (IRAK, NIK, IKK, p38 or MAP kinase inhibitor, etc.), IL, -1β converting enzyme inhibitor, TNFa converting enzyme (TACE) inhibitor), T cell signal inhibitor (kinase inhibitor, metalloproteinase inhibitor, sulfasalazine, azathioprine, 6-mercaptopurine, angiotensin converting enzyme inhibitor, soluble cytokine receptor (Eg, soluble p55 or p75 TNF receptor and derivatives p75 TNFRIgG (Embrel (ENBREL ™) and p55 TNFRIgG (Lunacept), sIL-lRI, sIL-IRII, sIL-6R), Anti Disease cytokines (such as IL-4, IL-10, IL-12, IL-13 and TGFP), celecoxib, folic acid, hydroxychloroquinine sulfate, rofecoxib, etanercept, infliximab, naproxen, valdecoxib, sulfasalazine, methylprednisolone, meloxicam, acetic acid Methylprednisolone, gold sodium thiomalate, aspirin, triamcinolone acetonide, propoxyfennapsylate / apap, folate, nabimeton, diclofenac, piroxicam, etodolac, diclofenac sodium, oxaprozin, oxycodone HC1, hydrocodone bitartrate / apapodiclo, / Misoprostol, fentanyl, anakinra, human recombinant, tramadol HC1, Salsalate, sulindac, cyanocobalamin / fa / pyridoxine, acetaminophen, sodium alendronate, prednisolone, morphine sulfate, lidocaine hydrochloride, indomethacin, glucosamine, sulfate / chondroitin, amitripterin HC1, sulfadiazine, oxycodone HC1 / acetaminophen, olopatadine HC1, misoprostol, naproxen sodium, omeprazole, cyclophosphamide, rituximab, IL-1 TRAP, MRA, CTLA4-IG, IL-18 BP, anti-IL-18, anti-IL15, BIRB-796, SCIO- 469, VX-702, AMG-548, VX-740, roflumilast, IC-485, Soliasis C-801 and mesoprum. Suitable antimicrobial agents include, but are not limited to, quaternary ammonium salts such as benzalkonium chloride.

Some of the excipient materials described herein may have more than one function in the formulation. For example, the substance may be both a solvent and a transdermal absorption enhancer, or both a solvent and a carrier. The above material classifications are not to be construed as limiting or limiting in any way.

The present application provides in another embodiment a pharmaceutical composition in any dosage form suitable for topical administration, comprising Apremilast as an active agent. The composition is preferably in the form of a solution, suspension, dispersion, emulsion, cream, ointment, gel, lotion, foam, paste or spray.

Various topical delivery systems are well known and can be utilized to administer the compositions of the present invention (eg. liposome capsules, microparticles, microcapsules, etc.).

In the case of non-sprayable topical dosage forms, viscous to semi-solid or solid dosage forms corresponding to topical application, preferably comprising a carrier or one or more excipients, preferably having a greater mechanical viscosity than water, are used. Suitable dosage forms include, but are not limited to, solutions, suspensions, emulsions, creams, ointments, powders, salves, salves, and the like. If necessary, the dosage form can be sterilized or mixed with adjuvants (eg. preservatives, stabilizers, wetting agents, buffers or salts, etc.) to affect various properties (eg. osmotic pressure).

In some embodiments, the composition can be in the form of an emulsion. This emulsion may be an O / W (oil / water) type emulsion or a W / O (water / oil) type emulsion. Aqueous-based emulsions, such as O/W emulsions, often have lower viscosities than other types of emulsions and have higher storage stability. When applied to the skin, an O/W emulsion usually gives the same feeling of use as an aqueous material, and therefore has a good feeling on the skin.

Other suitable topical dosage forms include sprayable aerosol preparations. The active ingredient is packaged as a mixture with a pressurized volatile material (eg. a gaseous propellant such as freon) with a solid or liquid inert carrier or packaged in a squeeze bottle.

As used herein, “cream” means a viscous liquid or a semisolid emulsion of O / W or W / O. The cream base is water-washable and contains an oil phase, an emulsifier and an aqueous phase. W / O creams can be prepared with fatty alcohols, such as cetyl alcohol and cetostearyl alcohol, and suitable emulsifiers having properties similar to emulsifying waxes. O/W creams can be prepared using emulsifiers such as setocrogol emulsifying wax. Suitable properties include the ability to adjust the viscosity of the emulsion and the physico-chemical stability over a wide pH range. The water-soluble or water-miscible cream base may contain a preservative system, and may be buffered to maintain an acceptable physiological pH.

As used herein, “ointment” means a semi-solid preparation containing an active agent added to a fatty, waxy or synthetic base. Usually, ointments are based on petrolatum or other petrolatum derivatives. The ointment bases used specifically are those which provide the desired properties such as appropriate drug delivery and emollient properties, as known to those skilled in the art.

As used herein, a “gel” is a transparent, sticky, jelly-like semi-solid or solid, prepared by incorporating a high molecular weight polymer in an aqueous or alcoholic base. Alcoholic gels are often dry and cool, and non-alcoholic gels are more lubricious. In some embodiments, the gel dosage form comprises the same or similar components as the solution or dispersion and an additional gelling agent.

As used herein, “lotion” refers to a liquid or semi-liquid preparation in which solid particles containing the active agent are present in a water-based or alcohol-based. Lotions are usually solid dispersions and can include OW type oily liquid emulsions. Lotions are often a desirable dosage form because of the ease of applying a highly flowable composition. Generally, lotions contain a suspending agent to produce a dispersion, and a compound suitable for holding the active agent in contact with the skin, such as methylcellulose, sodium carboxymethylcellulose, and the like.

As used herein, “foam” means a preparation formulated to be removed from a pressurized aerosol can using an inert propellant via a suitable applicator. Excipients suitable for preparing foam bases include, but are not limited to, propylene glycol, emulsifying waxes, cetyl alcohol and glyceryl stearate.

“Paste” is a semi-solid dosage form in which the active agent is suspended in a suitable base. Pastes are classified as either fat pastes or pastes derived from single-phase aqueous gels based on the nature of the base. The base of the fat paste is usually petrolatum, hydrophilic petrolatum or the like. Pastes produced from single-phase aqueous gels usually contain carboxymethylcellulose or the like as a base.

As used herein, “spray” means dispensing the present composition from a dispensing system as a collection of droplets or a jet.

In one aspect, topical application of the composition forms a depot on the skin without forming an occlusive film. Thus, the active period of the active agent can be extended while allowing skin “breathing”.

In still another embodiment, the present invention methods of treating non-psoriatic skin diseases or disorders, characterized by dry, hardened, scaly or flaky skin and fissures, by topical application to an affected epidermal area of a subject a topical dosage form comprising Apremilast, and continuing the administration until symptoms of the said disorder are abated. Also the present invention provides topical compositions for use in the prevention, amelioration or treatment of skin diseases, particularly epidermal cracks or fissures on the soles of feet, primarily on heels.

In one embodiment, the topical compositions of the present application are useful for controlling epidermal cracks or fissures and can provide a moisturizing and / or softening effect at the site of application of the skin. In one embodiment, the composition reduces dryness associated with skin, non-psoriatic skin diseases or disorders. In yet another embodiment, the composition can be applied directly to epidermal cracks or fissures and also reduces inflammation, removes scales from the affected skin and heals the fissures.

The present invention, in other embodiments, provides relief of skin irritation, discomfort, itching and other symptoms due to various conditions.

In another aspect, the present invention discloses a safe and commercially viable preparation process for a topical composition of Apremilast that is therapeutically effective. The composition is a composition having sufficient stability to provide an acceptable shelf life.

The compositions of the present invention can be prepared by any of the processes and techniques known in the art. The present inventor has used different formulation procedures and various excipients, surfactants, solubility enhancers and emulsifiers in the oil and water phases for the purpose of developing stable, uniform, cosmetically acceptable compositions.

Yet, another aspect of the present invention also relates to methods of treating, preventing, and managing pathological conditions that cause cytokine storm such as influenza, respiratory tract infections and pneumonia caused by viruses, specifically, those belonging to the Paramyxoviridae, Orthomyxoviridae, Flaviviridae, Picomaviridae, and Coronaviridae family, by administrating a therapeutically or prophylactically effective amount of an Apremilast.

Administration of Apremilast resulting in the prevention and treatment of such infectious diseases and disease-related complications is an unexpectedly effective treatment and preventative therapy.

Apremilast herein, can be conveniently administered by any of the routes conventionally used for drug administration. Apremilast herein can also be administered at conventional dosages in combination with a known second therapeutically active compound.

Administration of Apremilast for treating, preventing, and managing pathological conditions that cause cytokine storm such as influenza, respiratory tract infections and pneumonia caused by viruses can be oral, systemic (e.g., transdermal, nasal or suppository), or parenteral (e.g., intramuscular, intravenous or subcutaneous), sublingual, topical. It may be by any of the accepted methods of administration for agents that provide similar utility, including but not limited to intraperitoneal, intrapulmonary, intravaginal, rectal, or intraocular. In some embodiments, Apremilast is administered orally or parenterally. In other embodiments, Apremilast is administered by the pulmonary route.

Pharmaceutical compositions or formulations include, for example, solids, semi-solids, liquids and aerosol dosage forms such as tablets, capsules, powders, liquids, suspensions, liposomes, microspheres, suppositories, aerosols, nano-emulsions, injections, osmotic active pumps, pills, transdermal (including electro-transport) patches, etc. for long-term and / or timed administration, pulsed administration at a predetermined rate, etc. It can also be administered in sustained or controlled release dosage forms. In certain embodiments, the composition is provided in a unit dosage form suitable for single administration of the correct dose.

Pulmonary drug delivery methods include but not limited to metered and non-metered dose inhalers, dry powder inhalers, nebulisers and the like.

Both invasive and non-invasive drug delivery routes such as but not limited to pulmonary, oral, buccal, nasal, vaginal, uterine, ocular and transdermal or combinations thereof are the means by which Apremilast is administered for the treatment of pathological conditions that cause cytokine storm as per the current scope of this application.

In another aspect of the present invention, the major physical properties of respiratory drug aerosol particles include but not limited to particle size (dimensions such as diameter), shape, diffusivity, density and the electro static charge of Apremilast or Apremilast compositions are covered in the scope of this application. Particle size of Apremilast or Apremilast compositions thereof are in the range of pico, nano or micro meter range; specifically in the range of nano particles of less than 5000 nm or less than 1000 nm or less than 500 nm or less than 100 nm or less than 10 nm. Shape of Apremilast or Apremilast compositions thereof are either round, spherical, irregular, smooth surface and the like.

Also provided are methods of preventing a respiratory infections caused by viruses, specifically, those belonging to the Paramyxoviridae, Orthomyxoviridae, Flaviviridae, Picomaviridae, and Coronaviridae family in a subject comprising administering to a subject at risk of, or predisposed to, acquiring a respiratory infection, Apremilast, alone or in combination with antimicrobial agent, or a pharmaceutically acceptable salt thereof.

The principles, preferred embodiments, and modes of operation of the invention have been described herein. The invention, which is intended to protect, is not to be construed as limited to the particular form disclosed, as these descriptions are to be regarded as illustrative rather than restrictive. Those skilled in the art can make modifications and changes without departing from the spirit of the present invention.

Hereinafter, the present invention will be further described with reference to Examples, but these are not to be construed as limiting the scope of the invention in any way. In particular, the process conditions are merely exemplary and can be readily modified by those of ordinary skill in the art.

### EXAMPLES

Example 1: Topical composition of Apremilast

| Component | %w/w |
|---|---|
| Apremilast | 0.01 |
| Ethyl alcohol | 92.2 |
| Isopropyl myristate | 7.3 |
| Sodium lauryl sulfate | 0.1 |
| Undecylenic acid | 0.3 |

Procedure:
a) Under agitation, dissolve Apremilast in a portion of ethyl alcohol.
b) While stirring, add isopropyl myristate and undecylenic acid to the solution of step (a).
c) Add the remaining amount of ethyl alcohol to the solution of step (b) and mix.

Example 2: Topical composition of Apremilast

| Component | %w/w |
|---|---|
| Apremilast | 0.5 |
| Ethyl alcohol | 73.5 |
| Water | 20.5 |
| Sodium iodide | 3.7 |
| Potassium iodide | 1.4 |
| Sodium thiosulphate | 0.4 |

Procedure:
a) Under stirring, dissolve Apremilast in a portion of ethanol.
b) Dissolve iodides and sodium thiosulfate in water.
c) While stirring, add the solution obtained in step (b) to the solution of step (a).
d) Add ethyl alcohol and water to the solution of step (c) to adjust volume to required level and mix.

Example 3: Topical composition of Apremilast

| Component | %w/w | %w/w |
|---|---|---|
| Apremilast | 2 | 4 |
| Ethyl alcohol | 74.74 | 6.15 |
| Isopropyl myristate | 12 | 10 |
| Transcutol | 3 | 9 |
| Mineral oil | 7.26 | 69.85 |
| Acrylic polymer | 1 | 1 |

Procedure:
a) Dissolve Apremilast in a mixture of isopropyl myristate, mineral oil and transcutol with stirring.
b) Dissolve the acrylic acid polymer in ethyl alcohol.
c) While stirring, add the solution obtained in step (b) to the solution of step (a).
d) Add ethyl alcohol and mineral oil to the solution of step (c) to adjust volume to required level and mix.

Example 4: Topical composition of Apremilast

| Component | %w/w |
|---|---|
| Apremilast | 10 |
| Polyethylene glycol / Ethylene glycol palmitostearate | 7.5 |
| Mineral oil | 7.06 |
| Oleyl polyoxyglycerides | 2.94 |
| Diethylene glycol monoethyl ether | 5 |
| Propyl paraben | 0.8 |
| Methyl paraben | 0.2 |
| Butylated hydroxyl toulene | 0.05 |
| Hydroxy ethyl cellulose | 0.1 |
| Xanthum gum | 0.01 |
| Water | 66.35 |

Procedure:
a) Mix polyethylene glycol, ethylene glycol palmitostearate, oleoyl polyoxyglyceride, and mineral oil and heat to about 50-70° C.
b) Under continuous stirring at about 50-70° C., mix propylparaben, methylparaben, butylated hydroxytoluene with liquid (a).
c) Mix Apremilast with diethylene glycol and monoethyl ether.
d) Mix material of step (c) with material of step (b).
e) Dissolve hydroxyethyl cellulose and xanthan gum in water.
f) Under continuous stirring at about 50-70° C., slowly add the oil phase of (d) to the water phase of (e)
g) (or vice versa).
h) Homogenize mixture (f) and cool at room temperature.

Example 5: Topical composition of Apremilast

| Component | %w/w |
|---|---|
| Apremilast | 4 |
| Ethyl alcohol | 45 |
| Caprylic and capric triglycerides (Crodamol™) | 50 |
| Oleic acid | 0.85 |
| Sodium lauryl sulfate | 0.1 |

Procedure:
a) Under agitation, dissolve Apremilast in a portion of ethyl alcohol.
b) Under stirring, add Crodamol ™ to the solution of step (a).
c) Add oleic acid to the solution of step (b) under stirring.
d) While stirring, add sodium lauryl sulfate to the solution of step (c).
e) Add the remaining amount of ethyl alcohol to the solution of step (d) and mix.

Example 6: Nasal spray composition of Apremilast

| Component | %w/w |
|---|---|
| Apremilast | 1% |
| Benzyl alcohol | 30% |
| Dehydrated alcohol | 45% |
| n-dodecyl beta-D-maltoside | 10% |
| Vitamin E TPGS | 14% |

### Procedure

a) Dissolve or disperse Apremilast as fine dispersion into the mixture of benzyl alcohol, dehydrated alcohol, n-dodecyl beta-D-maltoside and vitamin E TPGS.

Example 7: Nasal aerosol composition of Apremilast

| | |
|---|---|
| Component | %w/w |
| Apremilast | 0.1% - 5% |
| Benzyl alcohol | 5% - 10% |
| Dehydrated alcohol | 10% - 45% |
| HFA-134a (1,1,1,2 tetrafluoroethane) | Qs. |

### Procedure

a) Dissolve or disperse Apremilast as fine dispersion into the mixture of benzyl alcohol, dehydrated alcohol, and fill propellant into the canisters.

Example 8: Nasal spray composition of Apremilast

| Component | %w/w |
|---|---|
| Apremilast | 0.1% - 5% |
| Microcrystalline cellulose | 5% - 50% |
| Carboxymethylcellulose sodium | 10% - 15% |
| Polysorbate 80 | 1% - 5% |
| Dextrose | 10% - 40% |
| Benzalkonium chloride | 1% - 5% |
| Edetate sodium | 1% - 5% |

### Procedure

a) Disperse/dissolve the components Apremilast, microcrystalline cellulose, carboxymethylcellulose sodium, polysorbate, dextrose, benzalkonium chloride and edetate sodium as fine dispersion in water and pass it through high pressure homogeniser to get fine dispersion. Adjust the pH to obtain values between pH 4 and 8.

Example 9: Evaluation of physical parameters for the topical formulations
a) The topical formulations prepared according to examples 1 to 5 were subjected to various physical assessment parameters and the findings obtained were within the limits.
b) All the formulations showed good viscosity and spreadability.
c) All of the formulations showed desired thermal stability at 20° C., 30° C. and 40° C.

### Example 10: Evaluation Therapeutic Efficacy of the Topical Formulations

The therapeutic composition prepared according to example 1 was used to treat a 54 year old female suffering from heel fissures (cracked heels). The patient was known to experience a recurrence of heel fissures for a long time. Topical application of the composition for two days appeared to be effective in treating the fissures, as well as the inflammation and hardness of the skin associated with the condition (shown in FIG. -1(C)). Regular application of the formulation for 15 days resulted in complete healing of the fissures and also prevented further recurrence of the condition.

Having now fully described the invention, it will be apparent to one of ordinary skill in the art that many changes and modifications can be made thereto without departing from the spirit or scope of the invention as set forth herein.

We claim:

1. A pharmaceutical composition for the treatment of non-psoriatic skin diseases or disorders, wherein the said composition comprises:
a) Apremilast,
b) atleast one pharmaceutically acceptable carrier; and
c) one or more pharmaceutically acceptable excipients;
wherein said composition is suitable for topical application to an area of skin that is affected by the dermatological condition.

2. The pharmaceutical composition as claimed in claim 1, wherein the composition is effective in treating, preventing, and managing non-psoriatic skin diseases or disorders, characterized by dry, hardened, scaly or flaky skin and fissures.

3. The pharmaceutical composition as claimed in claim 1, wherein the composition is in the form of a solution, suspension, dispersion, emulsion, cream, ointment, gel, lotion, foam, paste or spray.

4. The pharmaceutical composition as claimed in claim 1, wherein the composition comprises 0.01 to about 30% w/w of Apremilast, 5 to 99% w/w of a carrier, 0.25 to 45% w/w of an emulsifier, and 0.001 to 15% w/w of a permeation enhancer.

5. A method of treating non-psoriatic skin diseases or disorders comprising the steps of applying an effective amount of a therapeutic composition to an area of skin that is affected by the dermatological condition, wherein the composition comprises:
a) Apremilast,
b) atleast one pharmaceutically acceptable carrier; and
c) one or more pharmaceutically acceptable excipients;
wherein said composition is suitable for topical application to an area of skin that is affected by the dermatological condition.

6. The method of treating non-psoriatic skin diseases or disorders as claimed in claim 2, wherein the method comprising applying an effective amount of composition to the affected area of skin twice a day.

7. The method of treating non-psoriatic skin diseases or disorders as claimed in claim 2, wherein the non-psoriatic skin diseases or disorders are characterized by dry, hardened, scaly or flaky skin and fissures.

8. The method of treating non-psoriatic skin diseases or disorders as claimed in claim 2, wherein the composition is in the form of a solution, suspension, dispersion, emulsion, cream, ointment, gel, lotion, foam, paste or spray.

9. The method of treating non-psoriatic skin diseases or disorders as claimed in claim 2, wherein the composition comprises 0.01 to about 30% w/w of Apremilast, 5 to 99% w/w of a carrier, 0.25 to 45% w/w of an emulsifier, and 0.001 to 15% w/w of a permeation enhancer.
